(19) United States
(12) Patent Application Publication
(10) Pub. No.: US 2013/0317483 A1
(43) Pub. Date: Nov. 28, 2013

(54) UNIVERSAL CONNECTOR FOR DRAINAGE OF BODILY FLUIDS

(71) Applicant: KIMBERLY-CLARK WORLDWIDE, INC., Neenah, WI (US)

(72) Inventors: Emily A. Reichart, Atlanta, GA (US); Nathan C. Griffith, Roswell, GA (US); Joseph A. Cesa, Cumming, GA (US); Lisa A. Dagnatchew, Towson, MD (US); Alison S. Bagwell, Alpharetta, GA (US)

(73) Assignee: KIMBERLY-CLARK WORLDWIDE, INC., Neenah, WI (US)

(21) Appl. No.: 13/707,753

(22) Filed: Dec. 7, 2012

Related U.S. Application Data
(60) Provisional application No. 61/651,075, filed on May 24, 2012.
(60) Provisional application No. 61/655,599, filed on Jun. 5, 2012.

(51) Int. Cl. A61M 27/00

(52) U.S. Cl. CPC A61M 27/00
USPC 604/541

(57) ABSTRACT

The present disclosure describes an adapter for use with two connectors in, for example, a pleural, peritoneal or other bodily fluid drainage system. The adapter allows the use of the disposal bag from one manufacturer to be used with the catheter of another. This simplifies the storage requirements of distributors and hospitals and reduces the everyday challenges faced by those patients requiring fluid drainage.

This application claims the benefit of priority from U.S. Provisional Application No. 61/651,075 filed on May 24, 2012 and from U.S. Provisional Application No. 61/655,599 filed on Jun. 5, 2012, the contents of which are incorporated herein by reference.

### BACKGROUND

The present disclosure relates to an apparatus and method for draining fluids from a body.

Fluid production in a part of the human body like the chest, abdomen or other area may be caused by disease, injury or as a result of surgery. The medical professional, a care giver or the patient if he is capable, may need to drain such effusion fluids that accumulate at the site of an injury to relieve pressure felt by the patient and to remove the excess fluid that may harbor infection. Drainage from the thoracic cavity or pleural cavity surrounding the lungs, known as pleural drainage, is commonly performed by the patient at home or at work. Drainage may occur at any time interval as determined by the patient's level of discomfort and may take from a few minutes to an hour.

In this system as illustrated in FIG. 1, a catheter 10 with a short length of tubing 12 is typically surgically installed in the pleural cavity with the other (distal) end of the tubing terminating outside the body. The catheter is usually installed under local anesthesia and is not changed or removed unless it becomes infected or is no longer needed. When drainage of the fluid becomes necessary, a canister 14 is attached via tubing 16 to the distal end of the tubing that was surgically installed in the patient's chest via a set of connections 18. The fluid usually drains via gravity into the canister, though some products use an evacuated canister. Some systems use a disposable plastic bag or other container in place of the drainage canister.

Various manufacturers compete in the marketplace to produce in-dwelling catheters, tubing connectors and canisters or bags for pleural drainage. These products are incompatible with the products of the other manufacturers. Bard Medical produces the BARD® Channel Drain. Carefusion sells the PleurX® catheter system that uses vacuum (an evacuated canister) to remove fluids.

The incompatibility of the products produced by manufacturers poses a problem for users who must ensure that they have the proper type of bag on hand. This also creates a problem for distributors and hospitals since they must stock multiple types of bags so that they have the proper bag available for their customers.

What is needed is a way of connecting the bags of one manufacturer to the catheter tubing of other manufacturers in order to simplify the storage requirements of distributors and hospitals, and to reduce the everyday challenges faced by those patients requiring pleural drainage.

### SUMMARY

The present disclosure describes an adapter connector for use with pleural, peritoneal or other bodily fluid drainage disposal bags. The adapter allows the bag of one manufacturer to be connected to and used with the in-dwelling catheters of other manufacturers. This simplifies the storage requirements of distributors and hospitals and reduces the everyday challenges faced by those patients requiring fluid drainage.

Other objects, advantages and applications of the present disclosure will be made clear by the following detailed description of a preferred embodiment of the disclosure and the accompanying drawings wherein reference numerals refer to like or equivalent structures.

### BRIEF DESCRIPTION OF THE DRAWINGS

FIG. 1 is a drawing of a patient having a catheter for drainage placed in the chest, tubing leading from the chest to a drainage bag, and connectors between the two for disconnecting the bag from the catheter tubing.

FIG. 2 is a drawing of the catheter end (left side piece) and bag end (right side piece) connectors from Bard Medical just before they are connected together. The Bard medical connector has a set of tines on the bag end connector that is held by a notch or ledge on the catheter end connector.

FIG. 3 is a drawing of the catheter end and bag end connectors from Bard Medical when they are connected together. The tines of the bag end connector have spread slightly and slipped over the ledge on the catheter end connector to hold the two connectors firmly together.

FIGS. 4A-D are drawings of the catheter end and bag end connectors from Bard Medical when they are connected together, when viewed from different angles and cross-sectional views. FIG. 4A is a cross sectional view along the widest part of the catheter end connector along the axis indicated in FIG. 4C. FIG. 4B is a cross sectional view at 90 degrees to FIG. 4A, along the axis indicated in FIG. 4D. FIG. 4C is an exterior view of the orientation of the connectors in FIG. 4B and FIG. 4D is an exterior view of the orientation of the connectors in FIG. 4A.

FIG. 5 is a drawing of the catheter end (left side piece) and bag end (right side piece) connectors from Carefusion just before they are connected together.

FIG. 6 is a drawing of the catheter end and bag end connectors from Carefusion when they are connected together

FIG. 7 is a drawing of only the bag end connector from Carefusion.

FIGS. 8A-D are drawings of a connector on the catheter end of a fluid drainage system from Kimberly-Clark. FIG. 8A is a perspective view of the catheter end connector showing the distal and proximal ends. FIG. 8B is a cross sectional view along the widest part of the catheter end connector along the axis indicated in FIG. 8D. FIG. 8C is a cross sectional view at 90 degrees to FIG. 8B along the axis indicated in FIG. 8E. FIG. 8D is an exterior view of the orientation of the connectors in FIG. 8C and FIG. 8E is an exterior view of the orientation of the connectors in FIG. 8B.

FIGS. 9A-C are drawings of a connector on the bag end of a fluid drainage system from Kimberly-Clark. FIG. 9A is a perspective view of the connector showing the distal end and proximal end. The central stem and tines are also clearly visible. FIG. 9B is a cross sectional view of the connector and FIG. 9C is a top view of the connector looking down on the tine at one side.

FIGS. 10A-E are drawings of the catheter end (left side piece) and bag end (right side piece) connectors from Kimberly-Clark when they are connected together. FIG. 10A is a perspective view of the two connectors when they are attached to each other. FIG. 10B is a cross sectional view along the narrowest part of the connectors along the axis indicated in FIG. 10 D. FIG. 10C is a cross sectional view at 90 degrees to FIG. 10B along the axis indicated in FIG. 10 E. FIG. 10D is an exterior view of the orientation of the connectors in FIG. 10C and FIG. 10E is an exterior view of the orientation of the connectors in FIG. 10B.

FIGS. 11A-E are drawings of an adapter that can be used between the connectors on the catheter end and the bag end of a fluid drainage system. FIG. 11A is a perspective view of the adapter showing the distal end and proximal end with its elongated portion. FIG. 11B is a cross sectional view of the adapter along the axis indicated in FIG. 11D. FIG. 11C is a cross sectional view at 90 degrees to FIG. 11B along the axis indicate in FIG. 11E. FIG. 11D is an exterior view of the orientation of the adapter in FIG. 11C and FIG. 11E is an exterior view of the orientation of the adapter in FIG. 11B.

FIGS. 12A-D are drawings showing the interaction of the adapter (left side piece) and the connector on the bag end (right side piece) of a fluid drainage system from Kimberly-Clark. FIG. 12A is a perspective view of the adapter and bag end connector when they are attached to each other. FIG. 12B is a cross sectional view of the adapter and connector along the narrowest part of the connector along the axis indicated in FIG. 12D. FIG. 12C is a cross sectional view at 90 degrees to FIG. 12B along the axis indicated in FIG. 12E. FIG. 12D is an exterior view of the orientation of the adapter and connector in FIG. 12C and FIG. 12E is an exterior view of the orientation of the adapter and connector in FIG. 12B.

FIG. 13 is a drawing showing the interaction of the adapter (center piece) and the connector on the catheter end (left side piece) of a fluid drainage system from Carefusion with a connector on the bag end (right side piece) of a fluid drainage system from Kimberly-Clark.

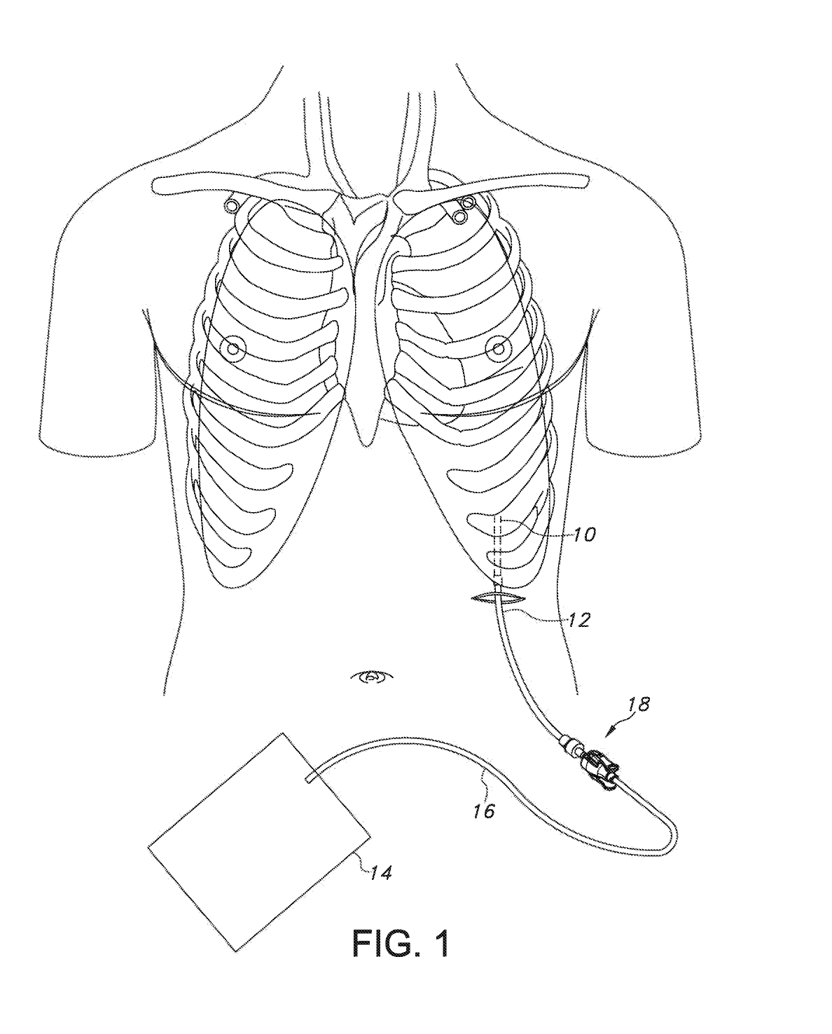

### DETAILED DESCRIPTION

Reference will now be made to the drawings in which the various elements of the present disclosure will be given numeral designations and in which the disclosure will be discussed so as to enable one skilled in the art to make and use the disclosure. It is to be understood that the following description is only exemplary of the principles of the present disclosure, and should not be viewed as narrowing the pending claims. Those skilled in the art will appreciate that aspects of the various embodiments discussed may be interchanged and modified without departing from the scope and spirit of the disclosure.

A number of different and incompatible styles of connectors are used for pleural, peritoneal or other fluid drainage. These different connectors have common features however, that allow them to be used in a similar manner. The connector on the catheter end, the end that remains connected to the patient, must have a valve, usually a one-way or “check” valve, to stop liquid from draining when the connector is not connected to a drainage bag connector. The connector on the bag end must have a means for opening the one-way valve so that liquid can flow between the two and into the bag. The connectors must be able to connect to tubing and of course must have a channel or cannula through their bodies for liquid to flow. After these criteria have been met the connectors may be very different.

The connectors that manufacturers have independently developed are of different shapes and sizes. These differences make it impossible or at least quite difficult to use, for example, the bag of one manufacturer with the catheter of another.

Examples of different catheter end and bag end connectors and adapters are shown in the Figures and described below.

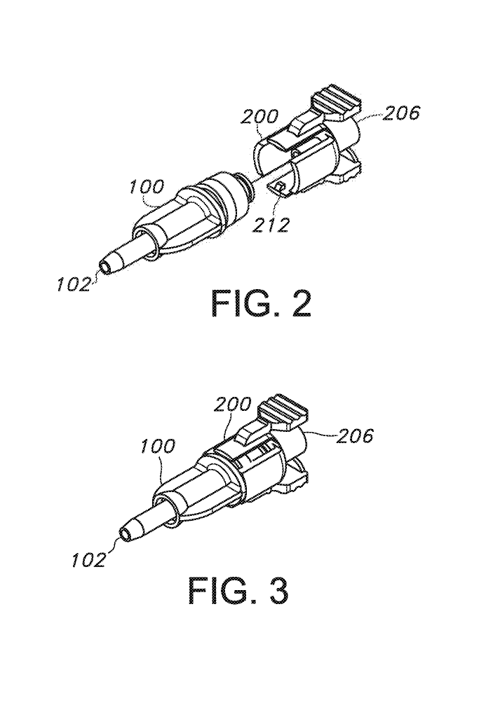

FIG. 2 is a drawing of a set of connectors from Bard Medical showing the bag end connector 200 and catheter end connector 100 immediately before they are connected together. The Bard medical connector has a set of tines 212 on the bag end connector 200 that is held by a notch or ledge 112 on the catheter end connector 100.

FIG. 3 is a drawing similar in nature to FIG. 2 but after the connectors 100, 200 have been connected together. The tines 212 of the bag end connector 200 have spread slightly and slipped over the ledge 112 on the catheter end connector 100 to hold the two connectors firmly together.

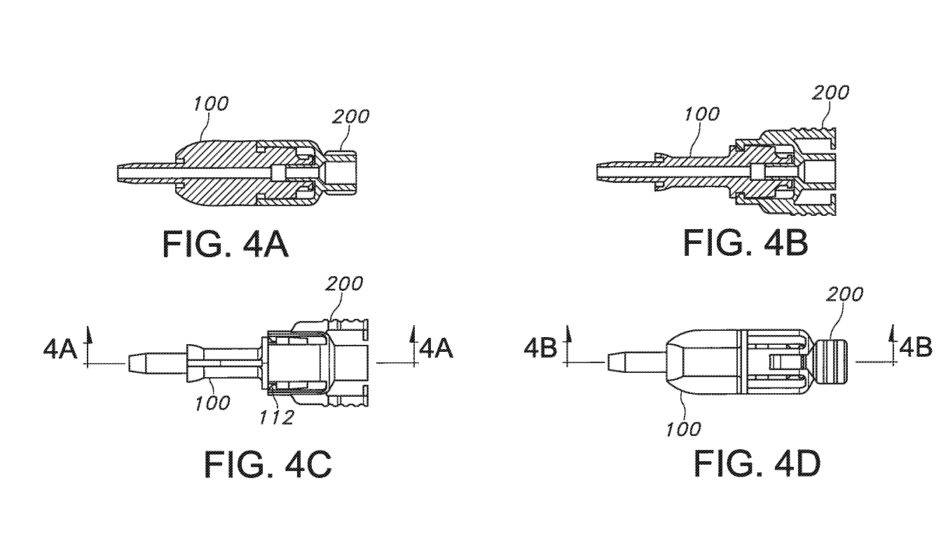

FIGS. 4A-D are drawings of the connected Bard Medical connectors from various views. FIG. 4A is a cross sectional view along the widest part of the catheter end connector 100 along the axis indicated in FIG. 4C. FIG. 4B is a cross sectional view at 90 degrees to FIG. 4A, along the axis indicated in FIG. 4D. FIG. 4C is an exterior view of the orientation of the connectors in FIG. 4B and FIG. 4D is an exterior view of the orientation of the connectors in FIG. 4A.

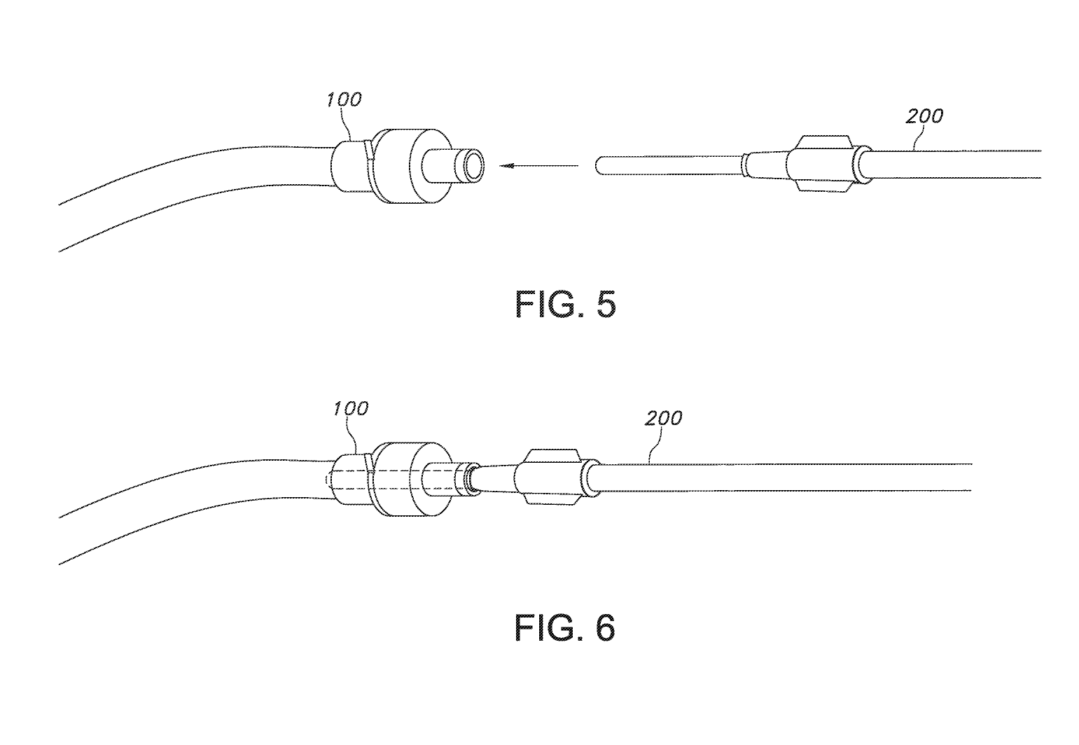

FIG. 5 is a drawing of the catheter end 100 and bag end 200 connectors from Carefusion just before they are connected together.

FIG. 6 is a drawing of the catheter end 100 and bag end 200 connectors from Carefusion when they are connected together. According to literature from Carefusion, there is an audible click when the two connectors are joined, indicating the presence of a locking mechanism.

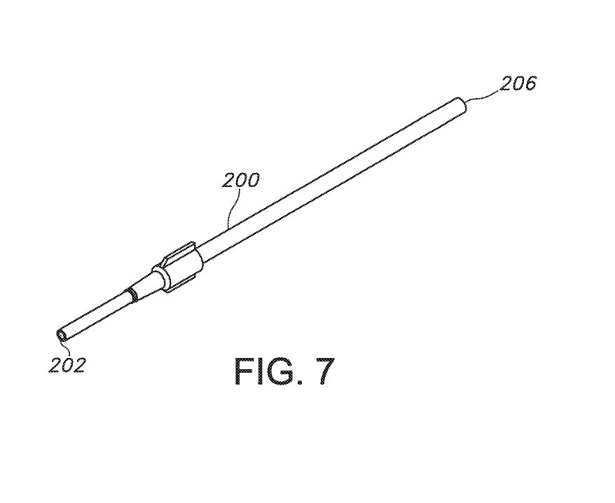

FIG. 7 is a drawing of only the bag end connector 200 from Carefusion showing a proximal end 202 for connection to the catheter end connector 100 and a distal end 206 for connection to tubing.

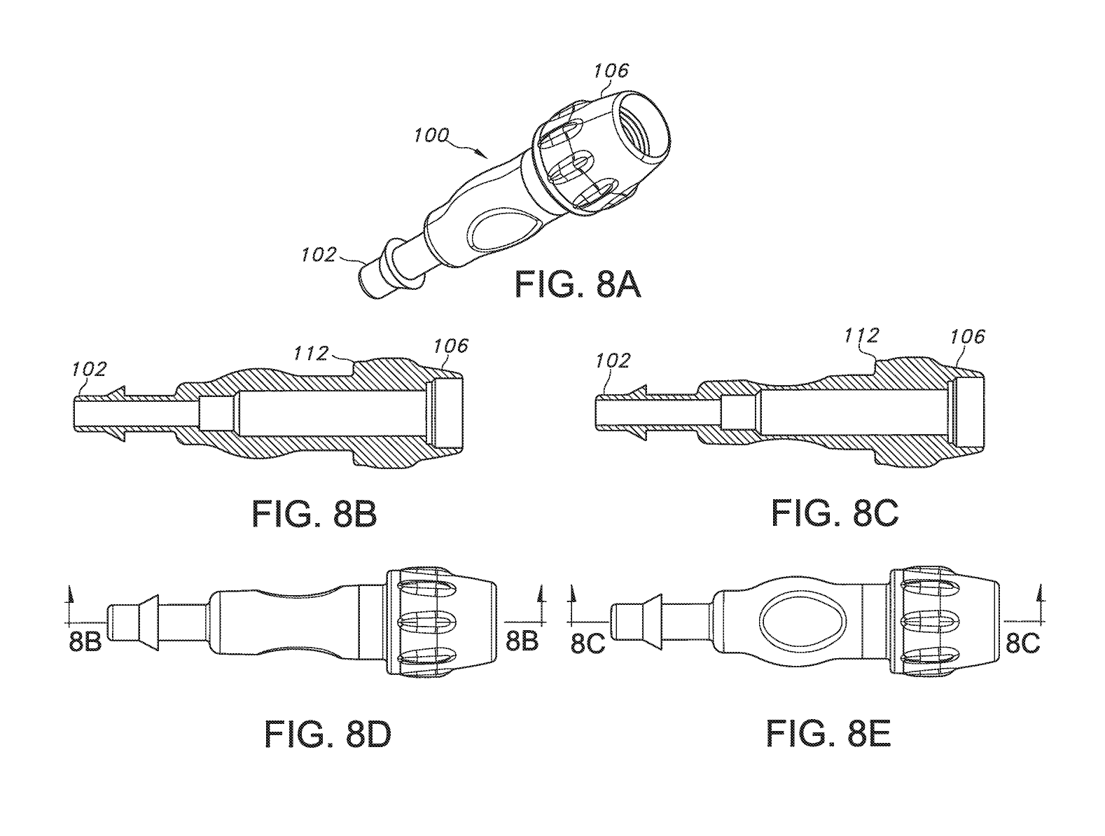

FIGS. 8A-D are drawings of a connector on the catheter end 100 of a fluid drainage system from Kimberly-Clark. FIG. 8A is a perspective view of the catheter end connector 100 showing the distal 106 and proximal ends 102. FIG. 8B is a cross sectional view along the widest part of the catheter end connector 100 along the axis indicated in FIG. 8D. FIG. 8C is a cross sectional view at 90 degrees to FIG. 8B along the axis indicated in FIG. 8E. FIG. 8D is an exterior view of the orientation of the connectors in FIG. 8C and FIG. 8E is an exterior view of the orientation of the connectors in FIG. 8B.

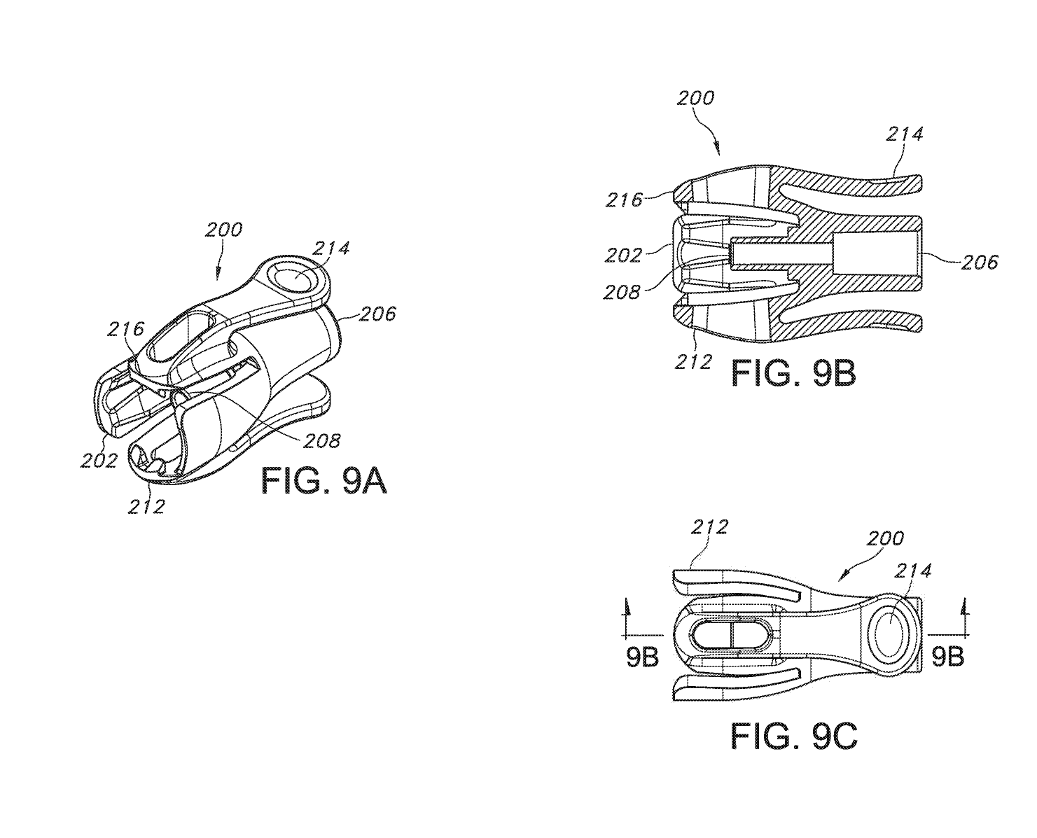

FIGS. 9A-C are drawings of a connector on the bag end 200 of a fluid drainage system from Kimberly-Clark. FIG. 9A is a perspective view of the connector showing the distal end 206 and proximal end 202. The central stem 208 and tines 212 are also clearly visible. FIG. 9B is a cross sectional view of the connector and FIG. 9C is a top view of the connector looking down on the tine 212 at one side.

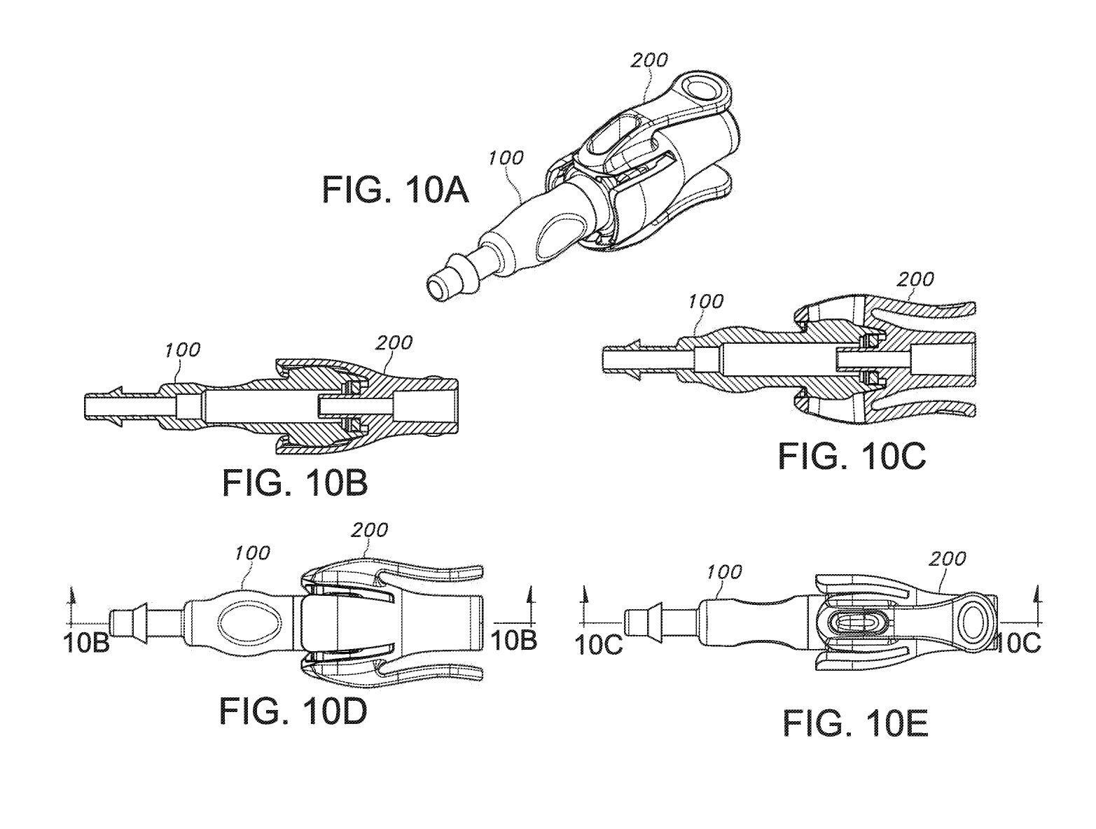

FIGS. 10A-E are drawings of the catheter end 100 and bag end 200 connectors from Kimberly-Clark when they are connected together. FIG. 10A is a perspective view of the two connectors when they are attached to each other. FIG. 10B is a cross sectional view along the narrowest part of the connectors along the axis indicated in FIG. 10D. FIG. 10C is a cross sectional view at 90 degrees to FIG. 10B along the axis indicated in FIG. 10 E. FIG. 10D is an exterior view of the orientation of the connectors in FIG. 10C and FIG. 10E is an exterior view of the orientation of the connectors in FIG. 10B.

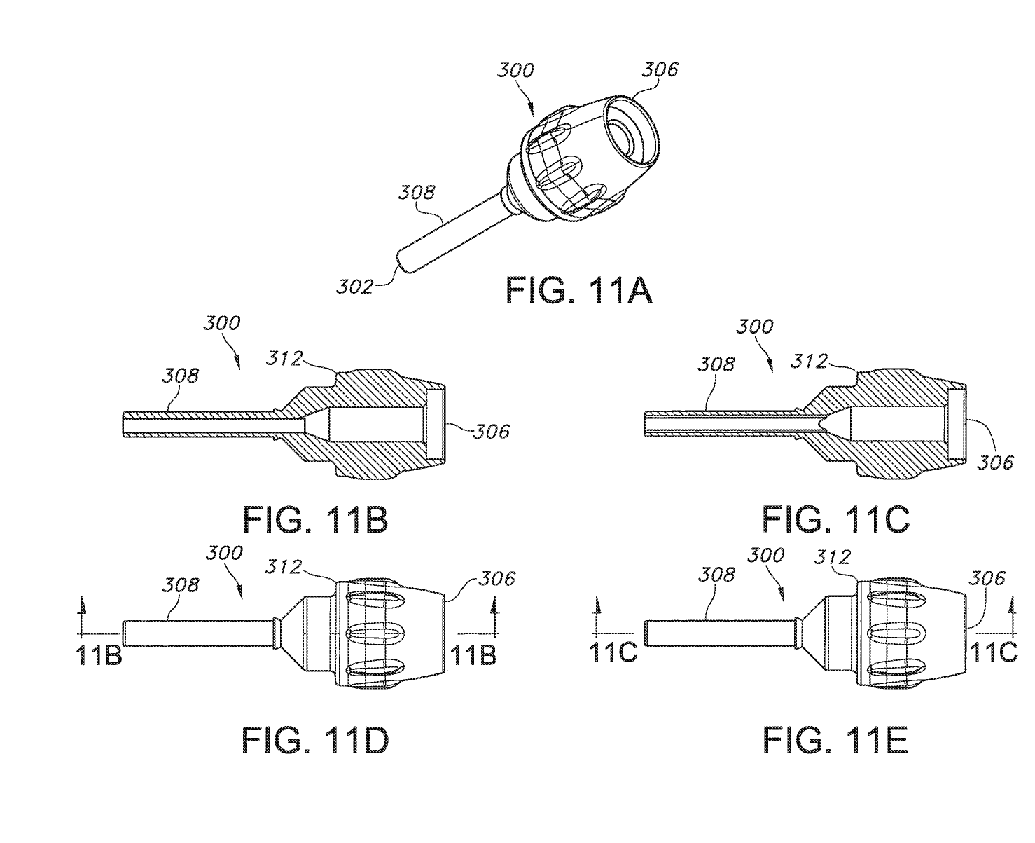

FIGS. 11A-E are drawings of an adapter that can be used between the connectors on the catheter end and the bag end of a fluid drainage system. FIG. 11A is a perspective view of the adapter showing the distal end 306 and proximal end 302 with its elongated portion 308. FIG. 11B is a cross sectional view of the adapter 300 along the axis indicated in FIG. 11D. FIG. 11C is a cross sectional view at 90 degrees to FIG. 11B along the axis indicate in FIG. 11E. FIG. 11D is an exterior view of the orientation of the adapter 300 in FIG. 11C and FIG. 11E is an exterior view of the orientation of the adapter 300 in FIG. 11B.

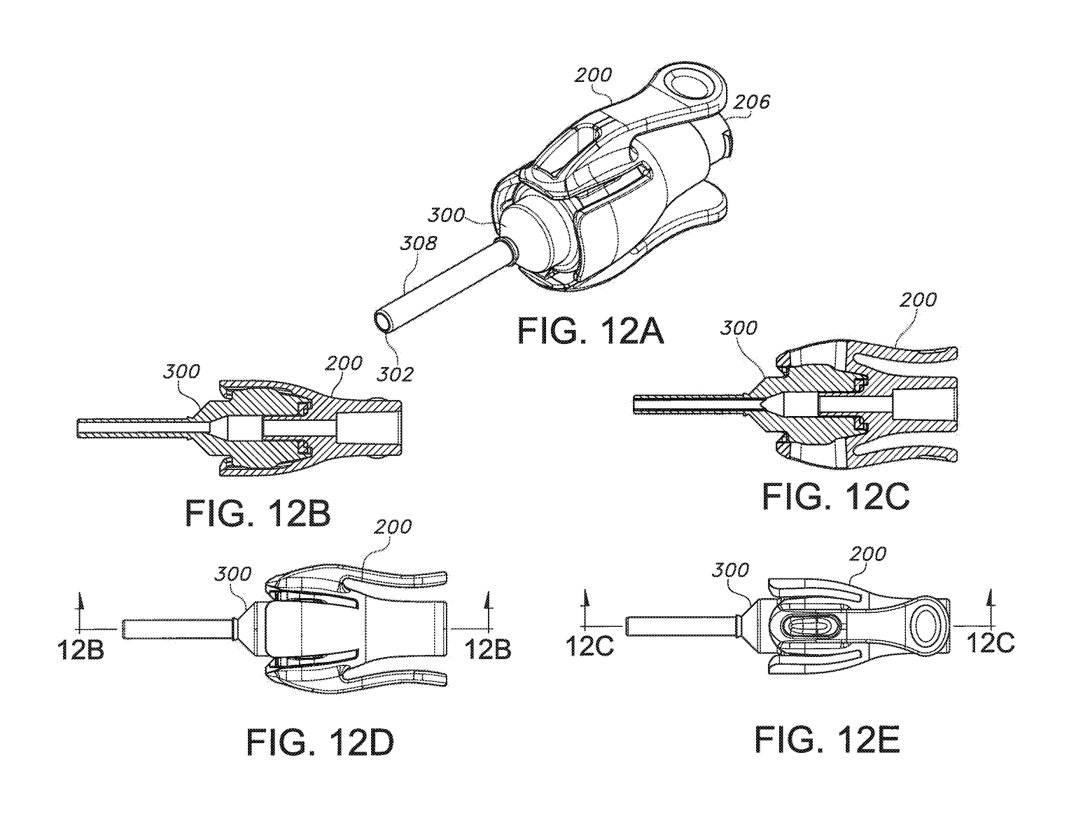

FIGS. 12A-D are drawings showing the interaction of the adapter and the connector on the bag end of a fluid drainage system from Kimberly-Clark. FIG. 12A is a perspective view of the adapter 300 and bag end connector 200 when they are attached to each other. FIG. 12B is a cross sectional view of the adapter 300 and connector 200 along the narrowest part of the connector 200 along the axis indicated in FIG. 12D. FIG. 12C is a cross sectional view at 90 degrees to FIG. 12B along the axis indicated in FIG. 12E. FIG. 12D is an exterior view of the orientation of the adapter 300 and connector 200 in FIG. 12C and FIG. 12E is an exterior view of the orientation of the adapter 300 and connector 200 in FIG. 12B.

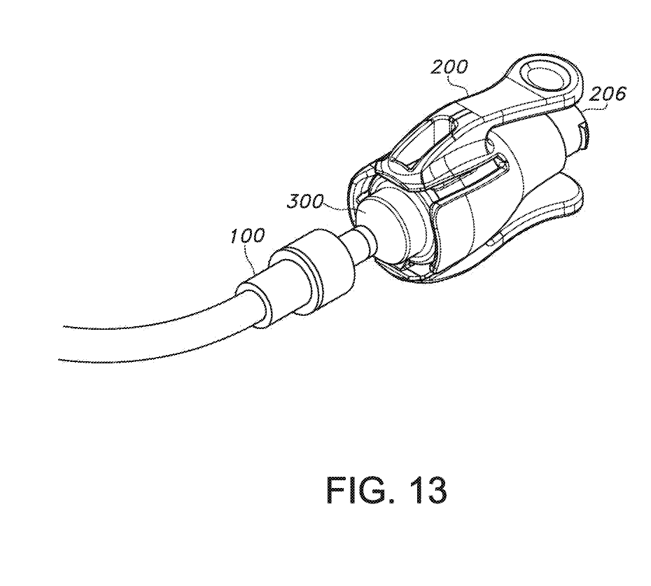

FIG. 13 is a drawing showing the interaction of the adapter 300 and the catheter end connector 100 of a fluid drainage system from Carefusion with a bag end connector 200 of a fluid drainage system from Kimberly-Clark.

The catheter end connectors 100 have a proximal end 102 for connecting to tubing 104 and a distal end 106 for connecting to the bag end connectors 200. In some embodiments the catheter end connector 100 has a circumferentially ledge 112 that is used to hold the tines 212 of the bag end connector 200.

In like manner, the bag end connectors 200 have a proximal end 202 that connects to the distal end 106 of the catheter end connector 100 and a distal end 206 that connects to tubing 204 that terminates in the collection bag 210. Some bag end connectors 200 have a central stem 208 that is inserted into the catheter end connector 100 to open the check valve within (not visible). The bag end connectors 200 have, in some embodiments, at least one set of tines 212 that are flexible enough to be spread apart slightly and then to spring back to approximately their original position. Finger pressure on the distal end 214 of the tines 212 is sufficient to separate them. The proximal end 216 of the tines 212 close on or latch onto the notch or ledge 112 of the complementary catheter end connector 100 to hold the catheter end connector 100 and bag end connector 200 together. The tines 212 may be easily unlatched from the ledge 112 by squeezing the distal end 214 of the tines 212 (opposite the ledge 112).

As can be gleaned from the drawings, the catheter end connectors 100 and bag end connectors 200 of each manufacturer are incapable of being effectively connected to the complementary connector of another manufacturer. In an emergency, the connectors of different manufacturers can be held together with the hands, or perhaps taped together, but this is not a satisfactory method under normal circumstances.

Disclosed herein is an adapter 300, as shown in FIG. 11, which may be used to connect the bag end connector 200 of one manufacturer to the catheter end connectors 100 of others. The adapter has a proximal end 302 that connects to the distal end 106 of the catheter end connector 100 of certain manufacturers, like for example the catheter end connector 100 shown in FIG. 5. The adapter 300 has an elongated portion 308 on the proximal end 302 that may be inserted into the catheter end connector 100 of certain manufacturers to function as a stem and open the check valve within (not visible).

In like manner, the adapter 300 has a distal end 306 that connects to the proximal end 202 of the bag end connector 200 of FIG. 9. The adapter also has a circumferential ledge 312 that may be used to latch the tines 212 of the bag end connector 200 of some embodiments.

In one embodiment, the adapter 300 has a total length of about 1.53 inches (3.88 cm), the outer width of the elongated portion 300 is about 0.12 inches (0.3 cm), the outer width of the distal portion is about 0.52 inches (1.33 cm) and the ledge 312 onto which the tines 212 of the bag end connector 200 latch are about 0.52 inches (1.32 cm) from the distal end 206. The width of the adapter at the inner part of the ledge is about 0.35 inches (0.9 cm).

FIG. 12 shows the interaction of the adapter 300 with a bag end connector 200 having tines 212 of FIG. 9. In use, the adapter 300 is first inserted into the bag end connector 200 until the tines 212 latch onto the ledge 312 of the adapter 300. The proximal end 302 of the adapter 300 is then inserted into the catheter end connector 100.

FIG. 13 shows the adapter 300 connected with the bag end connector 200 having tines 212 of FIG. 9 and the catheter end connector 100 of FIG. 5.

The connectors and adapters may be made from plastic materials. Suitable example materials include polyolefins, polyurethanes, nylons and the like.

As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

What is claimed is:

1. An adapter for use between two connectors, one of which has tines, comprising;
a proximal end for connection to a first connector and a wider distal end having a ledge for connection to a second connector having tines.

2. The adapter of claim 1 wherein the adapter has a total length of about 3.88 cm.

3. The adapter of claim 1 wherein said adapter has a narrow elongated portion on said proximal and an outer width of the elongated portion is about 0.3 cm.

4. The adapter of claim 1 wherein an outer width of the distal portion is about 1.33 cm.

5. The adapter of claim 1 wherein the ledge onto which the tines of the connector latch are about 1.32 cm from the distal end.

6. The adapter of claim 1 wherein the adapter has a width at the inner part of the is ledge of about 0.9 cm.

7. The adapter of claim 1 wherein the first end connector is a catheter end connector and the second connector is a bag end connector of a fluid drainage system.

8. A system for fluid drainage comprising a catheter end connector having an internal check valve, a bag end connector having tines, and an adapter between the two connectors that allows the bag end connector of one manufacturer to be used with the catheter end connector of another manufacturer.

9. A method of connecting the catheter end connector of one manufacturer to the bag end connector of another manufacturer using an adapter, comprising the steps of: inserting a distal end of said adapter into said bag end connector until tines on said bag end connector latch onto a ledge on said adapter and, inserting a proximal end of said adapter into said catheter end connector.
